(19) United States
(12) Patent Application Publication
(10) Pub. No.: US 2007/0078266 A1
(43) Pub. Date: Apr. 5, 2007

(54) Novel mercaptobenzimidazole substituted benzotriazole UV absorber and process for preparation thereof

(76) Inventors: Lonkar Sunil Parshuram, Maharashtra (IN); Singh Raj Pal, Maharashtra (IN); Tomer Namrata Singh, Aubiere Cedex (FR); Gardette Jean Luc, Aubiere Cedex (FR); Jestin Florence, Aubiere Cedex (FR); Lacoste Jacques, Aubiere Cedex (FR); Brocard Emmanuel, Saint-Yorre (FR)

Correspondence Address: LUEDEKA, NEELY & GRAHAM, P.C., P O BOX 1871, KNOXVILLE, TN, 37901, US

(21) Appl. No.: 11/376,973

(22) Filed: Mar. 16, 2006

(30) Foreign Application Priority Data
Sep. 30, 2005 (IN) 2631/DEL/2005

(51) Int. Cl. C07D 403/02

(52) U.S. Cl. 548/257

(57) ABSTRACT

The present invention provides a novel mercaptobenzimidazole substituted benzotriazole UV absorber having the general formula I 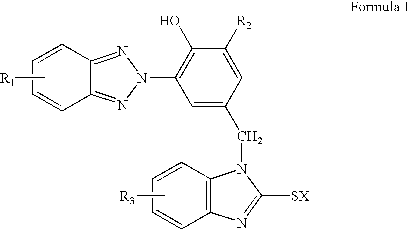
wherein R₁ is selected from the group consisting of hydrogen, halogen, C₁ to C₁₂ alkyl, linear or branched alkoxy group, R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and X is a hydrogen atom and a process for the preparation thereof.

### FIELD OF THE INVENTION

The present invention relates to a novel mercaptobenzimidazole substituted benzotriazole UV absorber having the general formula I 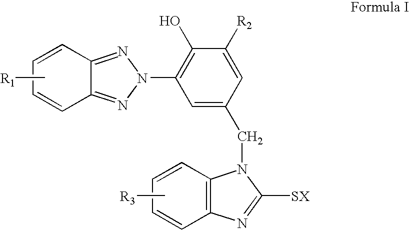
wherein R₁ is selected from the group consisting of hydrogen, halogen, C₁ to C₁₂ alkyl, linear or branched alkoxy group, R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and X is a hydrogen atom and a process for preparation thereof. More particularly it relates to the said compound and its preparation, using compound having formula (II): 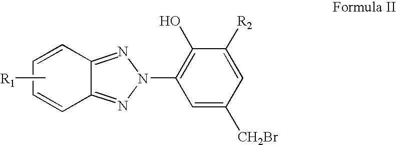
wherein, R₁ is selected from the group consisting of hydrogen, halogen, C₁ to C₁₂ alkyl, alkoxy (linear or branched); R₂ is selected from the group consisting of hydrogen, C₁-C₈ alkyl, cyclopentyl, cyclohexyl and cumyl and reacting it with a compound of formula (IV) 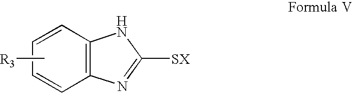
wherein, R₃ is a hydrogen atom or a linear alkyl groups particularly a methyl group or an ethyl group and X is a hydrogen atom.

The process for preparation of compound having formula (II) and its derivatives hvae been disclosed and claimed in our U.S. Pat. No. 6320056, November 2001

### BACKGROUND OF THE INVENTION

Polymers have replaced metals, glass, ceramics and papers in packaging, automobile, building construction, electronics, electrical coatings, furniture, pipes and heavy industrial equipments. In a nutshell, from agriculture to transport and aerospace to food packaging, the use of plastic has become an integral part of our daily life. Polymers, all natural and synthetic in common use, are susceptible to thermal/photo-oxidative degradation upon exposure to natural and artificial weathering.

Most thermoplastic polymers, elastomers and coating compositions deteriote mainly due to UV portion of natural light reaching to earth surface. The net result of this is degradation of the polymer, which leads to loss in the molecular weight and macroscopic physical properties. The preliminary effect of UV radiation on polymers is the formation of free radicals on the polymer chains, which reacts with the atmospheric oxygen. This results in the formation of the peroxide group, which causes the formation of carbonyl groups, and chain scission, simultaneously irradiation in the absence of oxygen, causes crosskinking. Ultimately this reflects in the mechanical properties and the color of the polymeric materials. In order to avoid this loss, different types of polymer stabilizers have been devised that protect the polymeric substrate from detrimental effect of light. Compatible and mobile stabilizers usually proved to be the best choice to attain the desired polymer stability. Most of these stabilizers are commercially available and are successfully employed single or in combination with other stabilizers for the effects of screener, antioxidant and thermal stabilizers. Depending upon the type of combination, the effect of stabilizer can be synergistic or antagonistic.

The efficiency of stabilizer depends on many factors viz. type of combination and proportion of stabilizers, compatibility with the polymer and molecular weight of the stabilizer. Benzotriazole based UV absorbers are one of the most important UV absorber and are used commercially. Antioxidants based on mercaptobenzimidazole are well known for their ability as hydrogen donating primary antioxidants and hydroperoxide decomposer secondary antioxidants and are commercially under use.

Mercaptobenzimidazole antioxidants and benzotriazole based UV absorbers are known to work in synergism and there is no literature on the synthesis of their coupled derivatives. Keeping in view all the above-mentioned requirements, the present invention provides novel benzotriazole UV absorber coupled with 2-mercaptobenzimidazole stabilizer for polymers. Following patents and literature provides information about synthesis of the photostabilizers and stabilization efficiency of UV absorber and mercapto antioxidant. European Patent No. EP0599269A1: November 1993, pertaining to the composition comprising sulphur containing derivatives of hydroxyphenylbenzotriazole, wherein sulphur functionality is utilized for improving the processing temperature of polymer specially resins and dose not point out the beneficial antioxidant properties of mercapto compounds. U.S. Pat. No. 5,942,626: August 1999 also described the thio substituted benzotriazole UV absorbers, here sulphur substitution is utilized to enhance the absorption range of benzotriazole and dose not mention any antioxidant nature of mercapto group. U.S. Pat. No. 5,319,091: June 1994 also disclose process for sulphur containing derivative of hydroxyphenyl benzotriazoles. Polymer Photochemistry, 1982, 2(1), 55-63 discloses the synergism of UV absorber and peroxide decomposer. JP02120341A2: October 1988 Antioxidant for Organic Polymer, also emphasize on the antioxidant utility of 2-mercaptobenzimidazole. In research article of J. Photochem Photobiology, 1991, 58 (3), 373-391 describes the hydrogen donating ability of mercapto compounds. Preparation and antioxidant nature of 2-mercaptobenzimidazole is disclosed in the patent JP54048764A2: September 1977 and in Acta. Chim. Solv. 2002, 49, 909-916. U.S. Pat. No. 4,317,759: March 1982 also discloses the stabilizing efficiency of 2-mercaptobenzimidazole. The importance of benzotriazoles as UV absorbers is commercially accepted and documented in past Progress in Polymer Science 2000, 25 (9), 1261-1335.

An online search using SCIFINDER and CHEMICAL ABSTRACT search engines did not provide any positive results for any molecule similar to that synthesized in this invention.

### OBJECTIVES OF THE INVENTION

The main objective of the present invention is to provide a novel mercaptobenzimidazole substituted benzotriazole UV absorber.

Another object of the present invention is to provide a process for the preparation of novel mercaptobenzimidazole substituted benzotriazole UV absorber.

Yet another object is to provide a process for the preparation of benzotriazole UV absorber coupled with 2-mercaptobenzimidazole antioxidant stabilizers and derivatives thereof.

This problem is being attempted in accordance with this invention by using in combination of two specific kinds of stabilizer compounds thereby its synergistic contributions. Moreover, these classes of combination are known to be compatible with polyolefins, diene elastomers, polycarbonates, polystyrene and can even be added in an additive proportion to obtain the desired degradation stability of various other polymer systems also.

### SUMMARY OF THE INVENTION

Accordingly the present invention provides a novel mercaptobenzimidazole substituted benzotriazole UV absorber having the general formula I 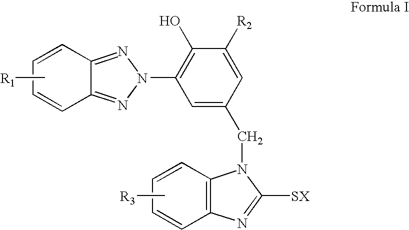
wherein R₁ is selected from the group consisting of hydrogen, halogen, C₁ to C₁₂ alkyl, linear or branched alkoxy group, R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and X is a hydrogen atom.

In an embodiment of the present invention the novel mercaptobenzimidazole substituted benzotriazole UV absorber obtained is selected from the group consisting of 2-[2′-hydroxy-5′-methylene(N-2-mercaptobenzimidazole)phenyl]benzotriazole, 2-[2′-hydroxy-5′-methylene(N-2-mercaptobenzimidazole)phenyl]5-chlorobenzotriazole, 2-[2′-hydroxy-3′-tert-butyl-5′-methylene(N-2-mercaptobenzimidazole)phenyl]benzotriazole and 2-[2′-hydroxy-5′-methylene(N-2-mercaptobenzimidazole)phenyl]-5′-methoxybenzotriazole.

The present invention further provides a process for the preparation of a novel mer captobenzimidazole substituted benzotriazole UV absorber having the general formula I 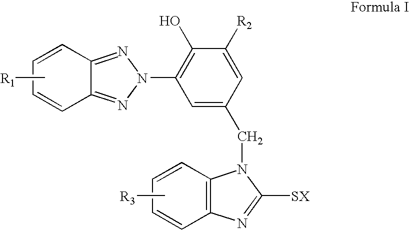
wherein R₁ is selected from the group consisting of hydrogen, halogen, C₁ to C₁₂ alkyl, linear or branched alkoxy group, R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and X is a hydrogen atom, the said process comprising the steps of:

a) dissolving a compound of formula IV 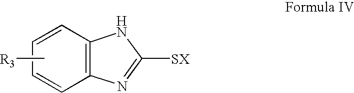
wherein, R₃ is a hydrogen atom or a linear alkyl group particularly a methyl group or an ethyl group and X is a hydrogen atom along with an organic base, in a dry organic solvent, under an inert atmospheric condition, agitating it initially for 20-30 minutes, adding thiol protecting group to the above said reaction mixture and stirring it for a period of 2-8 hrs, under inert atmosphere, filtering the insoluble mass from the above said reaction mixture and evaporating the solvent, followed by washing and re crystallizing the residue by known method to obtain the compound of formula III 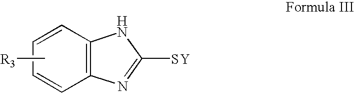
wherein, R₃ is hydrogen or a linear alkyl groups particularly a methyl group or an ethyl group and Y is selected from the group consisting of triphenyl methyl (trityl) group, benzyl chloride, acetyl benzyl chloride, 4-picolyl chloride, p-nitro benzyl chloride, diphenyl methyl chloride and diphenyl-4-pyridyl methyl chloride,

b) dissolving the compound of formula III obtained in step (a) and a compound of formula II, 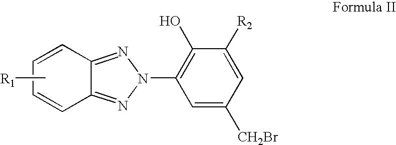
wherein, R₁ is selected from the group consisting of hydrogen, halogen, C₁ to C₁₂ alkyl, alkoxy (linear or branched); R₂ is is selectrd from the group consisting of hydrogen, C₁-C₈ alkyl, cyclopentyl, cyclohexyl and cumyl, separately in the same dry organic solvent, under inert condition, agitating the solution of the compound of formula III with an alkali metal hydroxide, for a period of 20-30 minutes, adding the solution of compound of formula II to the above said solution mixture of compound of formula III, gradually over a time period of 30-60 minutes, agitating the resultant reaction mixture for a period of 2-4 hrs, evaporating the organic solvent under pressure ranging from 400 to 500 mm Hg and dissolving the resultant solid mass in water and extracting the product in an organic solvent through repeated extraction and evaporating solvent to obtain the compound of formula V 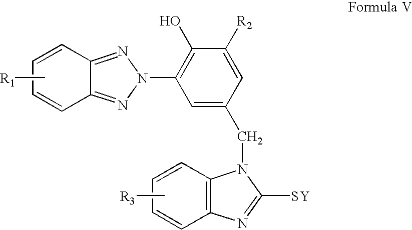
wherein, R₁ is selected from the group consisting of hydrogen, halogen, C₁ to C₁₂ alkyl, alkoxy (linear or branched), R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and Y is selected from the group consisting of triphenyl methyl (trityl) group, benzyl chloride, acetyl benzyl chloride, 4-picolyl chloride, p-nitro benzyl chloride, diphenyl methyl chloride and diphenyl-4-pyridyl methyl chloride,

c) treating the compound of formula V obtained in step (b) with a thiol deprotecting reagent, in an organic solvent, under stirring, a temperature in the range of 20-35° C., for a period of 1-3 hrs, followed by addition of water and extracting the product in an organic solvent, drying the solvent fraction with an anhydrous inorganic salt and neutralizing it with a base and evaporating the solvent to obtain the desired compound of formula (I).

In yet another embodiment the organic solvent used for dissolving the compound of formula IV in step (a) is selected from the group consisting of dimethylformamide, terahydrofuran, acetonitrile, 1,4-dioxane and dichloromethane.

In yet another embodiment the organic base used in step (a) is selected from the group consisting of trimethyl amine, imidazole, pyridine and 2,6-lutidine.

In yet another embodiment the thiol protecting group used in step (a) is selected from the group consisting of triphenyl methyl (trityl) group, benzyl chloride, acetyl benzyl chloride, 4-picolyl chloride, p-nitro benzyl chloride, diphenyl methyl chloride and diphenyl-4-pyridyl methyl chloride.

In yet another embodiment the organic solvent used for re crystallization of the crude mass is selected from the group consisting of dichloromethane, chloroform, acetone, diethyl ether and benzene.

In yet another embodiment the organic solvent used to dissolve compounds of formula III and formula II in step (b) is selected from the group consisting of terahydrofuran, 1,4 dioxane, dichloromethane, dimethylformamide and acetonitrile.

In yet another embodiment the metal hydroxide used in step (b) is selected from sodium hydroxide, potassium hydroxide and magnesium hydroxide.

In yet another embodiment the thiol-deprotecting agent used for de protecting the compound of formula V is selected from the group consisting of acetic acid, hydrofluoric acid, aqueous ammonia, sodium hydride and trifluroacetic acid.

In yet another embodiment the organic solvent used for extracting the product from aqueous medium in step(b) & step(c) is selected from the group consisting of dichloromethane, dichloroethane, ethyl acetate, diethyl ether and n-hexane.

In yet another embodiment the inert gas used is selected from nitrogen and argon.

In still another embodiment the inorganic base used to neutralize the solvent fraction containing the product in step (c) is selected from the group consisting of sodium carbonate sodium bicarbonate potassium carbonate and potassium bicarbonate.

The following examples are herein below given for the way of illustration and should not be construed to limit the scope of the present invention in any manner whatsoever.

### EXAMPLE 1

### Synthesis of 2-triphenylmethyl mercaptobenzimidazole

This compound was synthesized in dry and inert reaction conditions. In a 100 mL capacity round bottom flask 1 gms (0.006 moles) of 2-mercaptobenzimidazole (MBI) was dissolved in 20 mL of dry terahydrofuran (THF) along with 1.85 mL (0.0133 moles) of triethyl amine, simultaneously 1.856 gm (0.0071 mole) of triphenylmethyl chloride was added to the given reaction mixture. The reaction was stirred for 8 hrs under inert atmosphere of argon gas. After checking the TLC of the reaction mixture, the solid insoluble mass of triethylamine hydrochloride was removed by filtration. The residue was washed with several times with tetrahydrofuran, the mother liquor was evaporated under vacuum to give S-triphenylmethyl-mercaptobenzimidazole. The crude product weighed (2.8 gm) 82% yield. This was purified by recrystallization from benzene, off white colored crystals is resulted. The yield of the pure compound was 78% and its M.P=256° C.

### EXAMPLE 2

### Synthesis of 2-[2′-hydroxy-5′-methylene (N-2-triphenylmethylmercapto benzimida zole)phenyl]benzotriazole

This compound was synthesized under inert reaction condition. The compound S-triphenylmethyl mercaptobenzimidazole 1 gm (0.0022 mole) was taken in to 50 mL capacity round bottom flask and dissolved in excess of dry acetone under argon atmosphere with continuous stirring. After obtaining clear solution, powdered potassium hydroxide was added under inert atmosphere, the content of R.B was kept under vigorous stirring for 30 minutes to result reaction salt. In another 50 mL round bottom flask 2-(2′-hydroxy-5′-bromomethyl phenyl)benzotriazole 1.25 gm (0.0041mole) was taken and dissolved in 15 mL of dry acetone with stirring under argon atmosphere. The content of this R.B was added gradually to the first R.B over a period of 30-60 minutes. The color of the solution becomes yellow and as addition progressed, it turns to off white. The reaction mixture was further agitated for 2-4 hrs, the solvent in the R.B was removed by vaccuo under reduced pressure and the solid mass in the R.B was dissolved in 20 mL water and repeatedly extracted with dichloromethane (4×15 mL). Dichloromethane is then evaporated at 38° C. under vaccuo resulting a off white colored powdered product 2-[2′-hydroxy-5′-methylene(N-2-triphenylmethylmercapto benzimidazole)phenyl]benzotriazole. The crude yield was (1.69 gm) 84%. The product was recrystallized using an appropriate organic solvent to get 79% yield of pure product.

### EXAMPLE 3

### Synthesis of 2-[2′-hydroxy-5′-methylene(N-2-mercaptobenzinidazole)phenyl]benzotriazole

The compound 2-[2′-hydroxy-5′-methylene(N-2-triphenylmethylmercapto benzimidazole)phenyl]benzotriazole, 0.5 gm (0.00081 mol) was taken in R.B and refluxed with acetic acid/methanol (0.5:10 mL) for 2 hrs under inert atmosphere. The reaction mixture thus obtained was washed with water and extracted from dichloromethane (4×10 mL). The solvent was dried with anhydrous magnesium sulphate after neutralization with anhydrous potassium carbonate. Evaporation of the solvent results white colored product 2-[2′-hydroxy-5′-methylene(N-2-mercaptobenzimidazole)phenyl]benzotriazole with crude yield of 85%.

### EXAMPLE 4

### Synthesis of 2-[2′-hydroxy-5′-methylene(N-2-mercaptobenzimidazole)phenyl]-5-chlorobenzotriazole

The compound 2-[2′-hydroxy-5′-methylene(N-2-triphenylmethylmercapto benzimidazole)phenyl]-5-chlorobenzotriazole, 0.5 gm (0.00098 mol) was taken in R.B and refluxed with acetic acid/methanol (0.5:10 mL) for 2 hrs under inert atmosphere. The reaction mixture thus obtained was washed with water and extracted from dichloromethane (4×10 mL). The solvent was dried with anhydrous magnesium sulphate after neutralization with anhydrous potassium carbonate. Evaporation of the solvent results grayish colored product 2-[2′-hydroxy-5′-methylene(N-2-mercaptobenzimidazole)phenyl]5-chlorobenzotriazole with crude yield of 83%.

### EXAMPLE 5

### Synthesis of 2-[2′-hydroxy-3′-tert-butyl-5′-methylene(N-2-mercaptobenzimidazole)phenyl]benzotriazole

The compound 2-[2′-hydroxy-3-tert-butyl-5′-methylene(N-2-triphenylmethylmercapto benzimidazole)phenyl]benzotriazole, 0.5 gm (0.0012 mol) was taken in R.B and refluxed with acetic acid/methanol (0.5:10 mL) for 5 hrs under inert atmosphere. The reaction mixture thus obtained was washed with water and extracted from dichloromethane (4×10 mL). The solvent was dried with anhydrous magnesium sulphate after neutralization with anhydrous potassium carbonate. Evaporation of the solvent results off-white colored product 2-[2′-hydroxy-3′-tert-butyl-5′-methylene(N-2-mercaptobenzimidazole)phenyl]benzotriazole with crude yield of 85%.

### EXAMPLE 6

### Synthesis of 2-[2′-hydroxy-5′-methylene(N-2-mercaptobenzimidazole)phenyl]-5′-methoxybenzotriazole

The compound 2-[2′-hydroxy-3-tert-butyl-5′-methylene(N-2-triphenylmethylmercapto benzimidazole)phenyl]-5′-methoxybenzotriazole, 0.5 gm (0.0088 mol) was taken in R.B and refluxed with acetic acid/methanol (0.5:10 mL) for 5 hrs under inert atmosphere. The reaction mixture thus obtained was washed with water and extracted from dichloromethane (4×10 mL). The solvent was dried with anhydrous magnesium sulphate after neutralization with anhydrous potassium carbonate. Evaporation of the solvent results white colored product 2-[2′-hydroxy-5′-methylene(N-2-mercaptobenzimidazole)phenyl]-5′-methoxybenzotriazole with crude yield of 87%.

### ADVANTAGES OF THE PRESENT INVENTION ARE

1. The process is economical

2. The process comprises the commonly available reagents and employs mild reaction condition.

3. High yield (e 80%) can be achieved very easily.

4. Reaction can be carried out via very facile route with very simple and moderate reaction conditions.

We claim:

1. A novel mercaptobenzimidazole substituted benzotriazole UV absorber having the general formula I 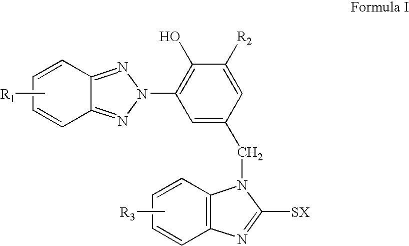
wherein R₁ is selected from the group consisting of hydrogen, halogen, C₁ to C₁₂ alkyl, linear or branched alkoxy group, R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and X is a hydrogen atom.

2. A novel mercaptobenzimidazole substituted benzotriazole UV absorber according to claim 1, obtained is selected from the group consisting of 2-[2′-hydroxy-5′-methylene(N-2-mercaptobenzimidazole)phenyl]benzotriazole, 2-[2′-hydroxy-5′-methylene(N-2-mercaptobenzimidazole)phenyl]5-chlorobenzotriazole, 2-[2′-hydroxy-3′-tert-butyl-5′-methylene(N-2-mercaptobenzimidazole)phenyl]benzotriazole and 2-[2′-hydroxy-5′-methylene(N-2-mercaptobenzimidazole)phenyl]-5′-methoxybenzotriazole.

3. A process for the preparation of a novel mercaptobenzimidazole substituted benzotriazole UV absorber having the general formula I 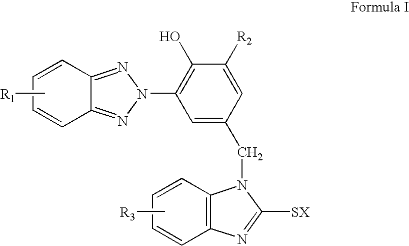
wherein R₁ is selected from the group consisting of hydrogen, halogen, C₁ to C₁₂ alkyl, linear or branched alkoxy group, R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and X is a hydrogen atom, the said process comprising the steps of:
a) dissolving a compound of formula IV 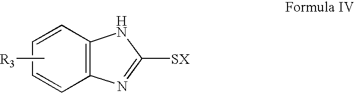
wherein, R₃ is a hydrogen atom or a linear alkyl group particularly a methyl group or an ethyl group and X is a hydrogen atom along with an organic base, in a dry organic solvent, under an inert atmospheric condition, agitating it initially for 20-30 minutes, adding thiol protecting group to the above said reaction mixture and stirring it for a period of 2-8 hrs, under inert atmosphere, filtering the insoluble mass from the above said reaction mixture and evaporating the solvent, followed by washing and re crystallizing the residue by known method to obtain the compound of formula III 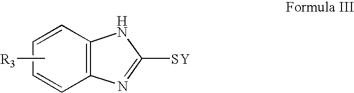
wherein, R₃ is hydrogen or a linear alkyl groups particularly a methyl group or an ethyl group and Y is selected from the group consisting of triphenyl methyl (trityl) group, benzyl chloride, acetyl benzyl chloride, 4-picolyl chloride, p-nitro benzyl chloride, diphenyl methyl chloride and diphenyl-4-pyridyl methyl chloride,
b) dissolving the compound of formula III obtained in step (a) and a compound of formula II, 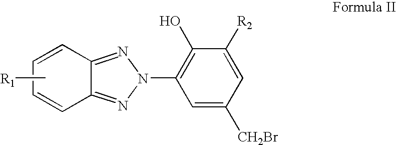
wherein, R₁ is selected from the group consisting of hydrogen, halogen, C₁ to C₁₂ alkyl, alkoxy (linear or branched); R₂ is is selectrd from the group consisting of hydrogen, C₁-C₈ alkyl, cyclopentyl, cyclohexyl and cumyl, separately in the same dry organic solvent, under inert condition, agitating the solution of the compound of formula III with an alkali metal hydroxide, for a period of 20-30 minutes, adding the solution of compound of formula II to the above said solution mixture of compound of formula III, gradually over a time period of 30-60 minutes, agitating the resultant reaction mixture for a period of 2-4 hrs, evaporating the organic solvent under pressure ranging from 400 to 500 mm Hg and dissolving the resultant solid mass in water and extracting the product in an organic solvent through repeated extraction and evaporating solvent to obtain the compound of formula V 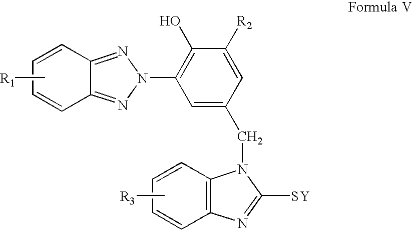
wherein, R₁ is selected from the group consisting of hydrogen, halogen, C₁ to C₁₂ alkyl, alkoxy (linear or branched), R₂ is selected from the group consisting of hydrogen, C₁-C₈ alkyl, cyclopentyl, cyclohexyl and cumyl, R₃ is a hydrogen atom or a linear alkyl group, particularly a methyl group or an ethyl group and Y is selected from the group consisting of triphenyl methyl (trityl) group, benzyl chloride, acetyl benzyl chloride, 4-picolyl chloride, p-nitro benzyl chloride, diphenyl methyl chloride and diphenyl-4-pyridyl methyl chloride,
c) treating the compound of formula V obtained in step (b) with a thiol deprotecting reagent, in an organic solvent, under stirring, a temperature in the range of 20-35° C., for a period of 1-3 hrs, followed by addition of water and extracting the product in an organic solvent, drying the solvent fraction with an anhydrous inorganic salt and neutralizing it with a base and evaporating the solvent to obtain the desired compound of formula (I).

4. A process according to claim 3, wherein the organic solvent used for dissolving the compound of formula IV in step (a) is selected from the group consisting of dimethylformamide, terahydrofuran, acetonitrile, 1,4-dioxane and dichloromethane.

5. A process according to claim 3, wherein the organic base used in step (a) is selected from the group consisting of trimethyl amine, imidazole, pyridine and 2,6-lutidine.

6. A process according to claim 3, wherein the thiol protecting group used in step (a) is selected from the group consisting of triphenyl methyl (trityl) group, benzyl chloride, acetyl benzyl chloride, 4-picolyl chloride, p-nitro benzyl chloride, diphenyl methyl chloride and diphenyl-4-pyridyl methyl chloride.

7. A process according to claim 3, wherein the organic solvent used for re crystallization of the crude mass is selected from the group consisting of dichloromethane, chloroform, acetone, diethyl ether and benzene.

8. A process according to claim 3, wherein the organic solvent used to dissolve compounds of formula III and formula II in step (b) is selected from the group consisting of terahydrofuran, 1,4 dioxane, dichloromethane, dimethylformamide and acetonitrile.

9. A process according to claim 3, wherein the metal hydroxide used in step (b) is selected from sodium hydroxide, potassium hydroxide and magnesium hydroxide.

10. A process according to claim 3, wherein the thiol-deprotecting agent used for de protecting the compound of formula V is selected from the group consisting of acetic acid, hydrofluoric acid, aqueous ammonia, sodium hydride and trifluroacetic acid.

11. A process according to claim 3, wherein the organic solvent used for extracting the product from aqueous medium in step(b)& step(c) is selected from the group consisting of dichloromethane, dichloroethane, ethyl acetate, diethyl ether and n-hexane.

12. A process according to claim 3, wherein the inert gas used is selected from nitrogen and argon.

13. A process according to claim 3, wherein the inorganic base used to neutralize the solvent fraction containing the product in step (c) is selected from the group consisting of sodium carbonate sodium bicarbonate potassium carbonate and potassium bicarbonate.

14. A process according to claim 3, wherein the mercaptobenzimidazole substituted benzotriazole UV absorber obtained is selected from the group consisting of 2-[2′-hydroxy-5′-methylene(N-2-mercaptobenzimidazole)phenyl]benzotriazole, 2-[2′-hydroxy-5′-methylene(N-2-mercaptobenzimidazole)phenyl]5-chlorobenzotriazole,
2-[2′-hydroxy-3′-tert-butyl-5′-methylene(N-2-mercaptobenzimidazole)phenyl]benzotriazole and 2-[2′-hydroxy-5′-methylene(N-2-mercaptobenzimidazole)phenyl]-5′-methoxybenzotriazole.
